(19) United States
(12) Patent Application Publication
(10) Pub. No.: US 2026/0276644 A1
(43) Pub. Date: Sep. 17, 2026

(54) MASS SPECTROMETRIC ANALYSIS

(71) Applicant: ELECTROPHORETICS LIMITED, Cobham, Surrey (GB)

(72) Inventors: Ian PIKE, Cobham, Surrey (GB); Karsten KUHN, Cobham, Surrey (GB)

(21) Appl. No.: 19/473,071

(22) PCT Filed: Apr. 11, 2024
(86) PCT No.: PCT/IB2024/053570

§ 371 Date: Oct. 6, 2025

Related U.S. Application Data
(60) Provisional application No. 63/458,629, filed on Apr. 11, 2023.

(51) Int. Cl. G01N 33/58; G01N 33/68

(52) U.S. Cl. CPC G01N 33/58; G01N 33/6848; G01N 2458/15

(57) ABSTRACT

Isotopic mass tags for labelling peptides and other biomolecules are provided as well as methods of detecting an analyte by identifying by mass spectrometry one or more of the mass labels or combinations thereof.

### TECHNICAL FIELD

This disclosure relates to a method of assaying a target analyte by mass spectrometry, particularly biomolecules such as nucleic acids and proteins. Specifically, the disclosure relates to a method of multiplexed mass spectrometry using sets of mass labels. Within a given set, the mass labels are isotopologues, meaning they have the same structural formula and differ only in their isotopic composition. As common in the literature, such isotopologue labels will be referred to as isotopic labels in this disclosure.

### BRIEF DESCRIPTION

The study of biological systems and particularly the understanding of human disease is dependent on the ability to detect changes caused in biological systems by or in response to a disease. Such changes provide means of diagnosis and offer insights into the targets for therapeutic compounds, such as vaccines and medicines. A wide range of biological molecules needs to be measured quantitatively to understand disease processes, including nucleic acids, proteins, steroids, sugars and lipids. In this context, the ability to quantitatively detect such biomolecules using mass spectrometers has provided considerable advances in their study and application to human and also to veterinary disease. The same advances have also occurred in environmental analysis and monitoring, and in food and beverage manufacturing. In particular, the use of stable isotopes to provide synthetic quantitative references have been developed in isotope dilution mass spectrometry for monitoring of all classes of biomolecules. However, these methods have traditionally required an available synthetic standard, which is not always possible.

Recently, a range of chemical mass tags bearing heavy isotope substitutions have been developed to further improve the quantitative analysis of biomolecules by mass spectrometry. Depending on the tag design, members of tag sets are either isotopic having the same chemical structure but different absolute masses, or isobaric (i.e., often even isotopomeric), having both identical structure and absolute mass, but differing in the exact placement of heavy isotopes. Isotopic tags are typically used for quantification in MS mode whilst isobaric tags must be fragmented in MS/MS mode to release reporter fragments with a unique mass.

An early example of isotopic mass tags was the Isotope-Coded Affinity Tags (“ICAT”) (Gygi, S. P. et al., (1999) Nature Biotechnology, 17, 994-999). The ICAT reagents are a pair of mass tags bearing a differential incorporation of heavy isotopes in one (heavy) tag with no substitutions in the other (light) tag. Two samples are labelled with either the heavy or light tag and then mixed prior to analysis by LC-MS. A peptide present in both samples will give a pair of precursor ions with masses differing in proportion to the number of heavy isotope atomic substitutions.

The ICAT method also illustrates ‘sampling’ methods, which are useful as a way of reconciling the need to deal with small populations of peptides to reduce the complexity of the mass spectra generated while retaining sufficient information about the original sample to identify its components. The ‘isotope encoded affinity tags’ used in the ICAT procedure comprise a pair of biotin linker isotopes, which are reactive to thiols, for the capture peptides comprising cysteine. Typically 90 to 95% or proteins in a proteome will have at least one cysteine-containing peptide and typically cysteine-containing peptides represent about 1 in 10 peptides overall so analysis of cysteine-containing peptides greatly reduces sample complexity without losing significant information about the sample. Thus, in the ICAT method, a sample of protein from one source is reacted with a ‘light’ isotope biotin linker while a sample of protein from a second source is reacted with a ‘heavy’ isotope biotin linker, which is typically 4 to 8 Daltons (“Da”) heavier than the light isotope. The two samples are then pooled and cleaved with an endopeptidase. The biotinylated cysteine-containing peptides can then be isolated on avidinated beads for subsequent analysis by mass spectrometry. The two samples can be compared quantitatively: corresponding peptide pairs act as reciprocal standards allowing their ratios to be quantified. The ICAT sampling procedure produces a mixture of peptides that still accurately represents the source sample while being less complex than MudPIT, but large numbers of peptides are still isolated and their analysis by LC-MS/MS generates complex spectra. With 2 ICAT tags, the number of peptide ions in the mass spectrum is doubled compared to a label-free analysis. Further isotopic tags, such as IPCL, mTRAQ and dimethyl labeling allow for sample multiplexing in MS1. Such isotopic labels increase the number of precursor ions as a function of the multiplexing level, i.e., a four-plex set of tags will create four times as many precursors and thus the amount of time needed for MS1-multiplexed data-dependent acquisition (DDA) analysis (see Mertins, P. et al. (2012) Molecular & Cellular Proteomics 11) with potential large impacts on the level of protein coverage. In practical terms, this has limited isotopic tag sets to maximally 4 members.

However, more recently, techniques for multiplexed analysis of proteins using isotopic labels have been developed using data independent acquisition (DIA) methods (e.g., plexDIA or mDIA) that increase throughput multiplicatively with the number of labels, without reducing proteome coverage or quantitative accuracy (Derks, Jason, et al. (2023) Nature biotechnology 41.1: 50-59, Thielert, Marvin et al., bioRxiv preprint doi: https://doi.org/10.1101/2022.12.02.518917).

### BRIEF DESCRIPTION

In view of the shortcomings of existing mass labels, there still remains a need for sets of isotopic tags for labelling peptides and other biomolecules that enable higher levels of sample multiplexing whilst retaining identical chromatographic retention times and providing unique mass-to-charge ratios for each version of the same peptide sequence. This is achievable within DIA strategies where relative quantification is performed at the MS1 level of tandem mass spectrometry whilst identification of sets of peptides with similar mass-to-charge are made using windows of defined widths ranging from 100 Da to less than 10 Da, using the isotopic mass labels described herein. To maximize the multiplexing levels whilst retaining sufficient resolution, each isotopic tag should be resolved from all other tags in the set by at least 0.003 Da, by 0.006 Da, and in some cases by at least 1 Da.

In a first general aspect, the present disclosure relates to a set of two or more isotopic mass labels, wherein each label comprises the formula:

$$X - L - M - Re$$

wherein X is a coding moiety having an exact mass, L is a bond optionally cleavable by collision in a mass spectrometer, M is a mass modifier, and Re is a reactive functionality for attaching the mass label to an analyte or the analyte, and X comprises the following general formula:

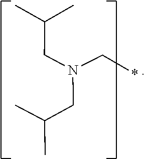

In other aspects, X comprises the general formula:

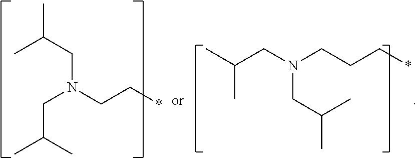

In some aspects, the present disclosure relates to a set of two or more isotopic mass labels, wherein each mass label comprises the formula:

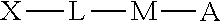

wherein X is a coding moiety having an exact mass, L is a bond optionally cleavable by collision in a mass spectrometer, M is a mass modifier, and A is the analyte, and wherein each mass label in the set has a different integer mass. In some aspects, X comprises the general formula:

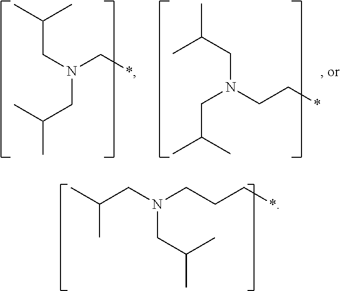

In some aspects, the set of two or more isotopic mass labels includes one or more mass labels, wherein the mass modifier M has the following structure:

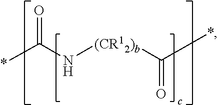

wherein:
   each R₁ is independently H, a substituted or unsubstituted straight or branched C₁-C₆ alkyl group, a substituted or unsubstituted aliphatic cyclic group, a substituted or unsubstituted aromatic group or a substituted or unsubstituted heterocyclic group or an amino acid side chain,
   a is an integer from 0-10,
   b is at least 1, and
   c is at least 1.

In some aspects, the set of two or more isotopic mass labels includes one or more mass labels wherein the mass modifier M has the following structure:

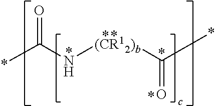

wherein:
   each R₁ is independently H, a substituted or unsubstituted straight or branched C₁-C₆ alkyl group, a substituted or unsubstituted aliphatic cyclic group, a substituted or unsubstituted aromatic group or a substituted or unsubstituted heterocyclic group or an amino acid side chain,
   a is an integer from 0-10,
   b is at least 1,
   c is at least 1, and
   wherein * is an isotopic mass adjuster moiety and represents that oxygen is ¹⁸O, carbon is ¹³C, nitrogen is ¹⁵N or hydrogen is ²H, and wherein one or more * may be present.

In some aspects, the set of two or more isotopic mass labels comprises mass labels having the following structure:

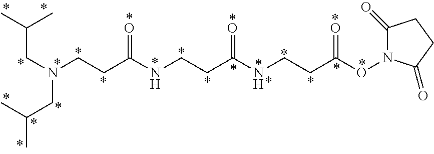 [SMILES: CC(C)CN(CCC(=O)NCCC(=O)NCCC(=O)ON1C(=O)CCC1=O)CC(C)C]

wherein * represents that oxygen is ¹⁸O, carbon is ¹³C, nitrogen is ¹⁵N, and hydrogen is ²H, and wherein one or more * may be present.

In some aspects, the set of two or more isotopic mass labels comprises n=6 mass labels having the following structures:

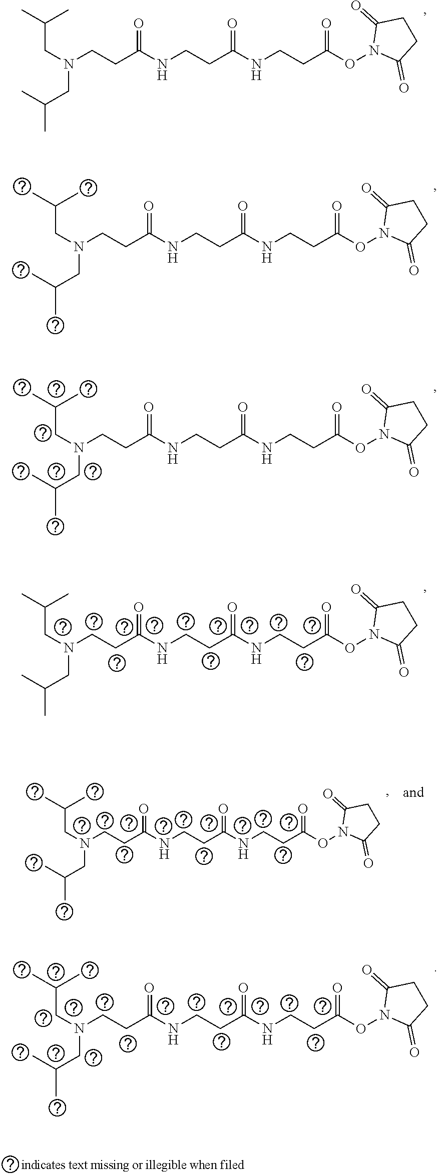 [SMILES: CC(C)CN(CCC(=O)NCCC(=O)NCCC(=O)ON1C(=O)CCC1=O)CC(C)C ; CC(C)CN(CCC(=O)NCCC(=O)NCCC(=O)ON1C(=O)CCC1=O)CC(C)C ; CC(C)CN(CCC(=O)NCCC(=O)NCCC(=O)ON1C(=O)CCC1=O)CC(C)C ; CC(C)CN(CCC(=O)NCCC(=O)NCCC(=O)ON1C(=O)CCC1=O)CC(C)C ; CC(C)CN(CCC(=O)NCCC(=O)NCCC(=O)ON1C(=O)CCC1=O)CC(C)C ; CC(C)CN(CCC(=O)NCCC(=O)NCCC(=O)ON1C(=O)CCC1=O)CC(C)C]

In some aspects, the difference in exact mass between at least two of the mass labels is less than 100 millidaltons (“mDa”), less than 50 mDa, or less than 20 mDa. In some aspects, the difference in exact mass between at least two of the mass labels is 2.5 mDa, 2.9 mDa, 6.3 mDa, 8.3 mDa, 9.3 mDa, or 10.2 mDa.

In another aspect, the present disclosure relates to a method of mass spectrometry analysis, which method comprises quantifying an analyte by identifying by mass spectrometry ions with a mass-to-charge ratio equivalent to the analyte mass modified by the addition of one member of the set of isotopic mass labels, wherein the mass label is a mass label from a set of mass labels according to the disclosure, and wherein the analyte identification is obtained from data-independent acquisition mass spectrometry.

In some aspects, a method of mass spectrometry analysis comprises: a) providing a plurality of samples, wherein each sample is differentially labelled with an isotopic mass label or a combination of isotopic mass labels, each selected from a set of two or more isotopic mass labels as described herein; b) mixing the plurality of labelled samples to form an analysis mixture comprising labelled analytes; c) detecting the labelled analytes in a mass spectrometer; d) isolating one or more labelled analytes in the mass spectrometer based on one or more isolation windows of ≥1 Da and then dissociating them to form labelled analyte fragments; e) detecting the analyte fragments; and f) identifying the one or more labelled analytes on the basis of the analyte fragments.

In some aspects, the one or more isolation windows comprise an isolation window that is ≤10 Da, ≤20 Da, ≤50 Da, and/or ≤100 Da.

In some aspects, the dissociation in step d) is collision-induced dissociation in a mass spectrometer. In some aspects, the dissociation is collision-induced dissociation in a mass spectrometer with a resolution of greater than 60,000 at a mass-to-charge ratio of 400, optionally a resolution of greater than 100,000 at a mass-to-charge ratio of 400, or a resolution greater than 250,000 at a mass-to-charge ratio of 400.

In some aspects, the labelled analytes may be identified on the basis of the MS2 spectrum of the labelled analyte fragments and quantified using the MS1 spectrum of the labelled analytes.

In some aspects, each isotopic mass label is resolved from all other tags in the set by at least 0.003 Da, by 0.006 Da, or at least 1 Da.

### BRIEF DESCRIPTION OF THE DRAWINGS

FIG. 1 is a reaction scheme showing the synthesis of 3-tert-butoxycarbonylamino propionic acid (Boc-βAla-OH).

FIG. 2 is a reaction scheme showing the synthesis of 3-aminopropionic acid benzyl ester hydrotosylate (H-βAla-OBn * pTosOH).

FIG. 3 is a reaction scheme showing the synthesis of benzyl 3-[3-(tert-butoxy carbonylamino)propanoylamino]propanoate (Boc-βAla-βAla-OBn).

FIG. 4 is a reaction scheme showing the synthesis of benzyl 3-(3-aminopropanoyl amino)propanoate hydrochloride (H-βAla-βAla-OBn*HCl).

FIG. 5 is a reaction scheme showing the synthesis of benzyl 3-[3-[3-(tert-butoxycarbonylamino)propanoylamino]propanoylamino]propanoate (Boc-βAla-βAla-βAla-OBn).

FIG. 6 is a reaction scheme showing the synthesis of benzyl 3-[3-(3-aminopropanoylamino)propanoylamino]propanoate hydrochloride (H-βAla-βAla-βAla-OBn*HCl).

FIG. 7 is a reaction scheme showing the synthesis of benzyl 3-[3-[3-(diisobutylamino)propanoylamino]propanoylamino]propanoate (diisobutyl-βAla-βAla-βAla-OBn).

FIG. 8 is a reaction scheme showing the synthesis of 3-{3-[3-(diisobutylamino)propionyl amino]propionylamino}propanoic acid (diisobutyl-βAla-βAla-βAla-OH).

FIG. 9 is a reaction scheme showing the synthesis of (2,5-dioxopyrrolidin-1-yl)3-[3-[3-(diisobutylamino)propanoylamino]propanoylamino]propanoate (diisobutyl-βAla-βAla-βAla-OSu).

FIG. 10 illustrates the full mass spectrum results for the set of 6 mass labels defined as Embodiment 1, showing a clean spectrum with no additional signals of relevant intensity and a visible mass shift of 4 Da between each successive mass label in the set.

FIG. 11 illustrates the mass spectrum results for the set of 6 mass labels defined as Embodiment 1, zoomed in to highlight the range of 400 to 500 Da.

FIGS. 12-17 illustrate the full mass spectrum results for each of the 6 mass labels defined as Embodiment 1, respectively.

FIG. 18 illustrates the relative elution profile of a tryptic peptide from bovine serum albumin labelled with an isobaric TMT® regent or an isotopic tag according to the present disclosure. The unlabeled peptide mobility is shown for reference. The peptide peak displays a good shape and no leading or trailing shoulders.

FIGS. 19-20 illustrate the MS/MS spectra of a tryptic peptide from bovine serum albumin either unlabeled (FIG. 19) or labelled with an isotopic label according to the present disclosure (FIG. 20). Sufficient structural ions are produced to allow confident sequence assignment.

FIG. 21 illustrates the charge state and relative signal intensity distributions for a complex proteome sample (HeLa digest (Thermo Scientific)) analyzed without labelling (upper panels) or when labelled with an isotopic label according to the present disclosure. The presence of the tag increases the percentage contribution of peptides with a 3+ charge state and also increases their average intensity.

FIG. 22 illustrates the rate of peptide identifications for 3+ charged isotopologue-tagged peptides is much higher than for other charge states and is at a similar level to typical identification rates in unlabeled proteomics experiments (comparative data not shown).

FIG. 23 illustrates the labelling efficiency for each member of a set of six-plex pD-iB-βAla tags in a HeLa digest sample measured by mass spectrometry. As shown, all tags have consistently high labelling efficiencies.

FIGS. 24-25 illustrate the consistency of retention time and signal intensity of tagged peptides from a HeLa digest at 33.3 minutes. This was seen to be the case throughout the elution gradient.

FIG. 26 illustrates examples of increased precursor ion complexity for three peptides eluting at 19.45 minutes of the elution gradient.

FIG. 27 illustrates two examples of increased fragment ion complexity for two DIA selection windows of 40 Da width.

FIG. 28 illustrates a diagram showing yeast protein abundance across the 6plex DIA Tag labelled HeLA/Yeast mixed proteome experiment with quantitative values being derived from MS1 precursor intensity values.

### DETAILED DESCRIPTION

### Sets of Mass Labels

The present disclosure provides sets of isotopic reactive tags having mass differences in the range of millidaltons to several Daltons for the purposes of labelling peptides and other biomolecules with potential multiplexing rates greatly in excess of 10-plex.

The present disclosure also provides for methods of use of isotopic reactive tags that enable novel forms of analysis of labelled peptides, proteins and other biological molecules, particularly for the discovery of biologically significant differences between sets of biological samples.

In a first aspect, the present disclosure relates to a set of two or more mass labels, wherein each mass label comprises the formula:

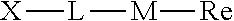

wherein:
X is a coding moiety having an exact mass,
L is a bond optionally cleavable by collision in a mass spectrometer,
M is a mass modifier, and
Re is a) a reactive functionality for attaching the mass label to an analyte or b) the analyte, wherein each mass label in the set has a unique exact mass, wherein the set comprises two or more mass labels, wherein each mass label has a coding moiety X comprising the following general formula:

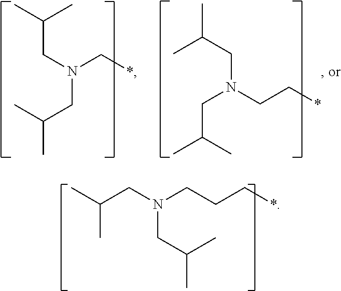

The term “exact mass” refers to the theoretical mass of the mass label or of the coding moiety and is the sum of the exact masses of the individual isotopes of the entire mass label or coding moiety, e.g., ¹²C=12.000000, ¹³C=13.003355 ¹H=1.007825, ¹⁶O=15.994915. The “exact mass” takes account of mass defects.

In the literature the term “isotopic” often refers to species that have different masses and are not co-selectable for MS/MS but in the context of this disclosure the term “isotopic” refers to species that have different masses but which can still be co-selected for MS/MS using DIA.

The difference in exact mass between at least two of the mass labels in a set may be less than 100 millidaltons (“mDa”), less than 50 mDa, or less than 20 mDa. In some aspects, the difference in exact mass between at least two of the mass labels in a set is 2.5 mDa, 2.9 mDa, 6.3 mDa, 8.3 mDa, 9.3 mDa, or 10.2 mDa due to common isotope substitutions. For example, if a first label comprises a ¹³C isotope, and in a second label this ¹³C isotope is replaced by ¹²C but a ¹⁴N isotope is replaced by a ¹⁵N isotope, the difference in exact mass between the two labels will be 6.3 mDa.

In the present specification, the term label is synonymous with the term tag.

It will be understood by the person with ordinary skill in the art that in order to achieve the desired masses for each tag within a set, one or both of the moieties X and M, the reactive functionality Re or the analyte may be modified with heavy isotopes. The heavy isotopes may be selected from ²H, ¹³C, ¹⁵N or ¹⁸O.

In some aspects, the aggregate molecular weight of the mass labels is 600 Da or less, e.g., 500 Da or less, 400 Da or less, or from 300 to 500 Da.

The mass labels according to the disclosure are designed to be reacted with a biomolecule, such as a protein to form a labelled biomolecule, e.g., a labelled protein.

In some aspects, the optionally cleavable bond L comprises, without limitation, an amide bond, a urea linkage, an ester linkage or an ether linkage. In some aspects, optionally cleavable bond L comprises an amide bond. In some aspects, the optionally cleavable bond L comprises a urea linkage. In some aspects, the optionally cleavable bond L comprises an ester linkage. In others, the optionally cleavable bond L comprises an ether bond. In some aspects, e.g., as illustrated by the mass label of Example 1, the optionally cleavable bond L comprises a carbon-carbon bond. In yet further aspects the optionally cleavable bond L is not cleavable by common fragmentation methods used in tandem mass spectrometry.

The term “mass modifier M”, as used herein, refers to a moiety that ensures that each mass label in the set has a desired mass. The mass modifier M is not necessarily to be detected by mass spectrometry. However, the mass modifier M may be detected as part of a complement ion (see below). The mass modifier M is not particularly limited structurally, but merely serves to vary the overall mass of the mass label.

In some aspects, the mass modifier M has the following structure:

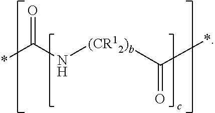

wherein:
each R₁ is independently H, a substituted or unsubstituted straight or branched C₁-C₆ alkyl group, a substituted or unsubstituted aliphatic cyclic group, a substituted or unsubstituted aromatic group or a substituted or unsubstituted heterocyclic group or an amino acid side chain,
a is an integer from 0-10,
b is at least 1, and
c is at least 1.

In some aspects, the mass modifier M is selected from:

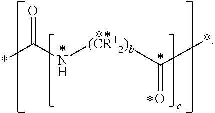

wherein:
each R₁ is independently H, a substituted or unsubstituted straight or branched C₁-C₆ alkyl group, a substituted or unsubstituted aliphatic cyclic group, a substituted or unsubstituted aromatic group or a substituted or unsubstituted heterocyclic group or an amino acid side chain,
a is an integer from 0-10,
b is at least 1,
c is at least 1, and
wherein * is an isotopic mass adjuster moiety and represents that oxygen is ¹⁸O, carbon is ¹³C, nitrogen is ¹⁵N or hydrogen is ²H, and wherein one or more * may be present.

In the mass labels according to the disclosure, Re may either be a reactive functionality for attaching the mass label to an analyte or be an analyte.

In some aspects, the mass tags additionally comprise a reactive functionality to allow the mass label to be conjugated to an analyte. The reactive functionality for attaching the mass label to the analyte is not especially limited and may comprise any appropriate reactive group.

The reactive functionality may react with an amino group on the biological molecule, for example, the ε-amino group of a lysine residue and the α-amino group of peptide or protein. In the simplest embodiments this may be an N-hydroxysuccinimide ester. Other reactive functionalities are contemplated herein such as those which react with thiol groups in biological molecules. In particular these reactive functionalities are designed to react with the thiol group of a cysteine residue. Examples of reactive groups of the present disclosure which are able to react with cysteine residues are the maleimido, haloacetyl and 2-dithiopyridine groups. The thiol group of cysteine undergoes nucleophilic addition across the double bond of the maleimido group and undergoes nucleophilic substitution with the haloacetyl or 2-dithiopyridine group.

Reactive functionalities which are capable of reacting with carbonyl or hydroxyl groups in biological molecules are also contemplated herein. In particular, these reactive functionalities are designed to react with the carbonyl or hydroxyl groups of steroid hormones. Reactive groups of the present disclosure which are able to react with carbonyl or hydroxyl groups in a biological molecule are hydrazide or —CONH—(CH₂)ₙ—ONH₂, wherein n is from 1 to 6 (e.g., wherein n is 3, i.e. aminoxypropyl amide). These groups react with carbonyl groups to form hydrazones or O-alkyloximes, respectively. Examples of reactive functionalities are shown in WO 2011/036059, which reference is incorporated herein.

In some aspects, the reactive functionality is an N-hydroxysuccinimide ester, a 2,3,5,6-tetrafluorophenyl ester or a sulphodichlorophenyl ester.

When Re is the analyte, the analyte may comprise, e.g., amino acids, peptides, polypeptides, nucleotides, oligonucleotides, polynucleotides, carbohydrates, lipids, phospholipids or combination thereof.

In some aspects, mass labels according to the disclosure, and sets of two or more mass labels comprising heavy isotope mass series modifying groups, are illustrated by exemplary Embodiment 1 below.

### Embodiment 1

The mass label has the structure:

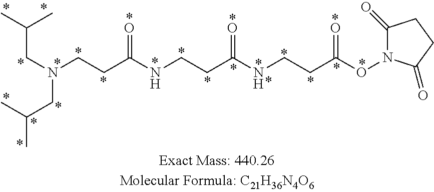 [SMILES: CC(C)CN(CCC(=O)NCCC(=O)NCCC(=O)ON1C(=O)CCC1=O)CC(C)C]

wherein * represents that oxygen is ¹⁸O, carbon is ¹³C, nitrogen is ¹⁵N, and hydrogen is ²H, and wherein one or more * may be present.

In some aspects, the mass modifier moiety * is ¹³C or ¹⁵N and the set comprises n=6 mass labels having the following structures:

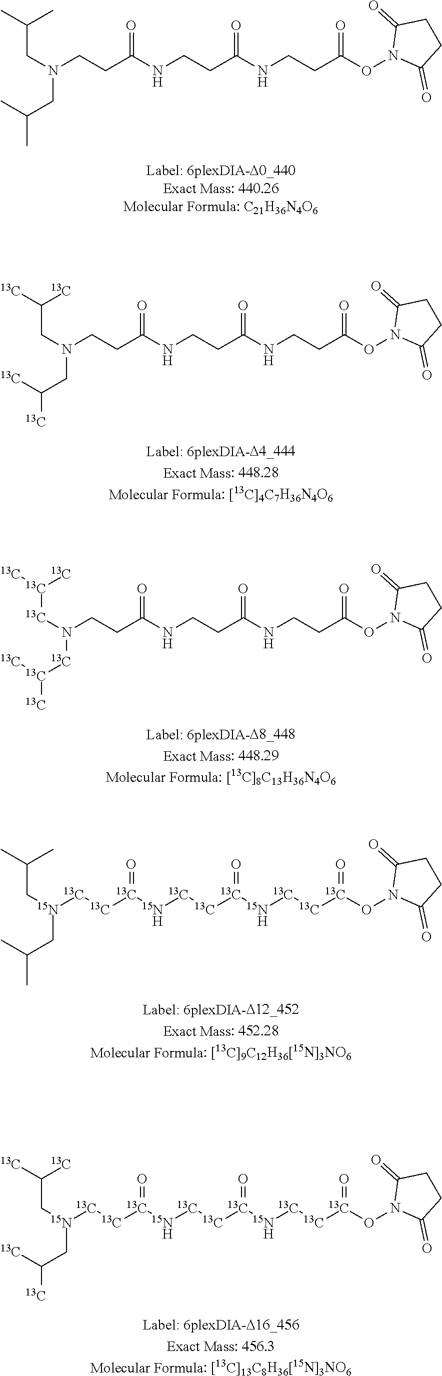 [SMILES: CC(C)CN(CCC(=O)NCCC(=O)NCCC(=O)ON1C(=O)CCC1=O)CC(C)C ; [13CH3]C([13CH3])CN(CCC(=O)NCCC(=O)NCCC(=O)ON1C(=O)CCC1=O)CC([13CH3])[13CH3] ; [13CH3][13CH]([13CH3])[13CH2]N(CCC(=O)NCCC(=O)NCCC(=O)ON1C(=O)CCC1=O)[13CH2][13CH]([13CH3])[13CH3] ; CC(C)C[15N](CC(C)C)[13CH2][13CH2][13C](=O)[15NH][13CH2][13CH2][13C](=O)[15NH][13CH2][13CH2][13C](=O)ON1C(=O)CCC1=O ; [13CH3]C([13CH3])C[15N](CC([13CH3])[13CH3])[13CH2][13CH2][13C](=O)[15NH][13CH2][13CH2][13C](=O)[15NH][13CH2][13CH2][13C](=O)ON1C(=O)CCC1=O]

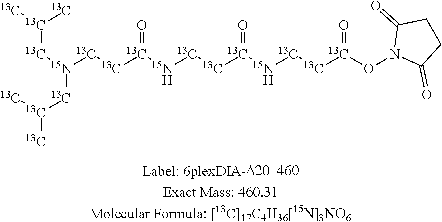 [SMILES: [13CH3][13CH]([13CH3])[13CH2][15N]([13CH2][13CH2][13C](=O)[15NH][13CH2][13CH2][13C](=O)[15NH][13CH2][13CH2][13C](=O)ON1C(=O)CCC1=O)[13CH2][13CH]([13CH3])[13CH3]]

### Embodiment 2

The mass label comprises any one of the following structures:

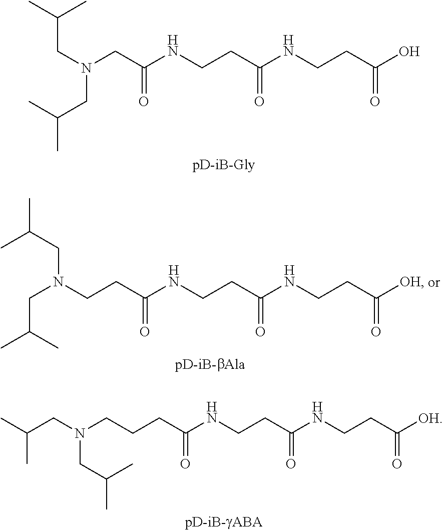 [SMILES: CC(C)CN(CC(=O)NCCC(=O)NCCC(=O)O)CC(C)C ; CC(C)CN(CCC(=O)NCCC(=O)NCCC(=O)O)CC(C)C ; CC(C)CN(CCCC(=O)NCCC(=O)NCCC(=O)O)CC(C)C]

In some aspects, a mass label may comprise any of the three structures shown above, wherein one or more oxygen, carbon, nitrogen, and/or hydrogen components is replaced by an isotopic substituent (e.g., as illustrated above with respect to Embodiment 1). These alternatives offer a range of multiplexing levels and different sizes.

### Methods of Mass Spectrometry Analysis

The present disclosure also provides for a method of mass spectrometry analysis, which comprise quantifying an analyte by identifying by mass spectrometry ions with a mass-to-charge ratio equivalent to the analyte mass modified by the addition of one member of the set of isotopic mass labels, wherein the mass label is a mass label from a set of mass labels according to the disclosure, and wherein the analyte identification is obtained from data-independent acquisition mass spectrometry.

In some aspects, a method of mass spectrometry analysis comprises: a) providing a plurality of samples, wherein each sample is differentially labelled with an isotopic mass label or a combination of isotopic mass labels, each selected from a set of two or more isotopic mass labels as described herein; b) mixing the plurality of labelled samples to form an analysis mixture comprising labelled analytes; c) detecting the labelled analytes in a mass spectrometer; d) isolating one or more labelled analytes in the mass spectrometer based on one or more isolation windows of ≥1 Da and then dissociating them to form labelled analyte fragments; e) detecting the analyte fragments; and f) identifying the one or more labelled analytes on the basis of the analyte fragments.

In some aspects, the one or more isolation windows comprise an isolation window that is ≤10 Da, ≤20 Da, ≤50 Da, and/or ≤100 Da.

In some aspects, the labelled analytes may be identified on the basis of the MS2 spectrum of the labelled analyte fragments and quantified using the MS1 spectrum of the labelled analytes.

In some aspects, the dissociation in step d) is collision-induced dissociation in a mass spectrometer. In some aspects, the dissociation is collision-induced dissociation in a mass spectrometer with a resolution of greater than 60,000 at a mass-to-charge ratio of 400, optionally a resolution of greater than 100,000 at a mass-to-charge ratio of 400, or a resolution greater than 250,000 at a mass-to-charge ratio of 400.

The analytes may be identified on the basis of: i) the mass spectrum of the labelled analytes; or ii) the mass spectrum of the mass labels and/or analyte fragments comprising an intact mass label. When identification according to ii) occurs, the analyte fragment optionally comprises a b-series ion comprising an intact mass label (e.g., a b1 ion).

Thus, in some aspects, the analytes may be identified on the basis of the mass spectrum of the labelled analytes.

In some aspects, the analytes may be identified on the basis of the mass spectrum of the mass labels and/or analyte fragments comprising an intact mass label. In some aspects, the analyte fragment comprising an intact mass label is a b-series ion comprising an intact mass label (e.g., a b1 ion).

For the purposes of resolving all of the possible tags described herein, mass spectrometers with high resolution are required but the nature of the instruments is not particularly important to the practice of this methods described herein. In addition, many of the tags that have been described in this application can still be resolved on instruments with only single dalton resolution as long as subsets of the possible tags that are separated by single dalton mass differences are used.

The mass labels and methods described herein are further explained by the following examples, which are merely illustrative and by no means limiting the scope of the invention claimed herein now or during prosecution of the present application.

### EXAMPLES

### Example 1: Synthesis of (2,5-Dioxopyrrolidin-1-yl)3-[3-[3-(diisobutylamino)propanoyl amino]-propanoylamino]-propanoate (Diisobutyl-βAla-βAla-βAla-OSu)

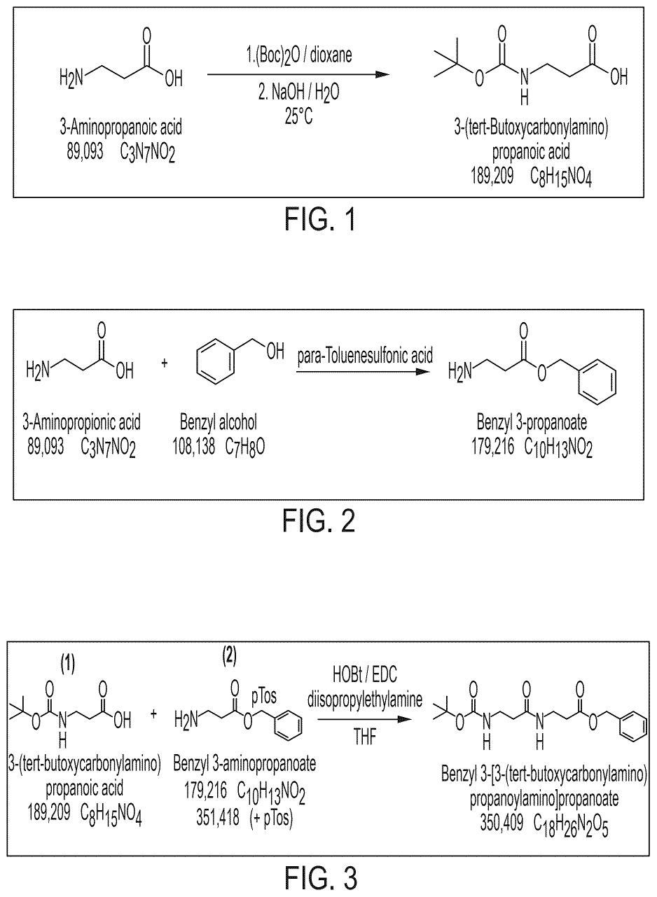

### Step 1: Synthesis of 3-tert-butoxycarbonylaminopropionic acid (Boc-βAla-OH) (see FIG. 1)

A solution of 16.32 g sodium hydroxide in 163 mL water is placed in the 2-L-two-neck flask. To this 15.0 g 3-aminopropanoic acid (D-alanine) and 150 mL dioxane are added. A turbid solution is formed. Then, 53.47 g di-tert-butyl-dicarbonate ((Boc)₂O) are added. An exothermic reaction (55-57° C.) is observed, the pH value settles at about 9-10 and a solid precipitates. Stirring is continued for 5 hours. Leave to stand overnight and add 250 mL water the next day so that a solution is formed. The reaction solution is shaken out twice with 250 mL diisopropyl ether. The diisopropyl ether phases are discarded. The aqueous phase is adjusted to pH 3 with 2M hydrochloric acid (approx. 150 mL) and the product is extracted 3 times with 200 mL dichloromethane. The organic phases are dried over magnesium sulphate, filtered, and concentrated to yield 29.78 g product (0.155 mol; 95.1%)

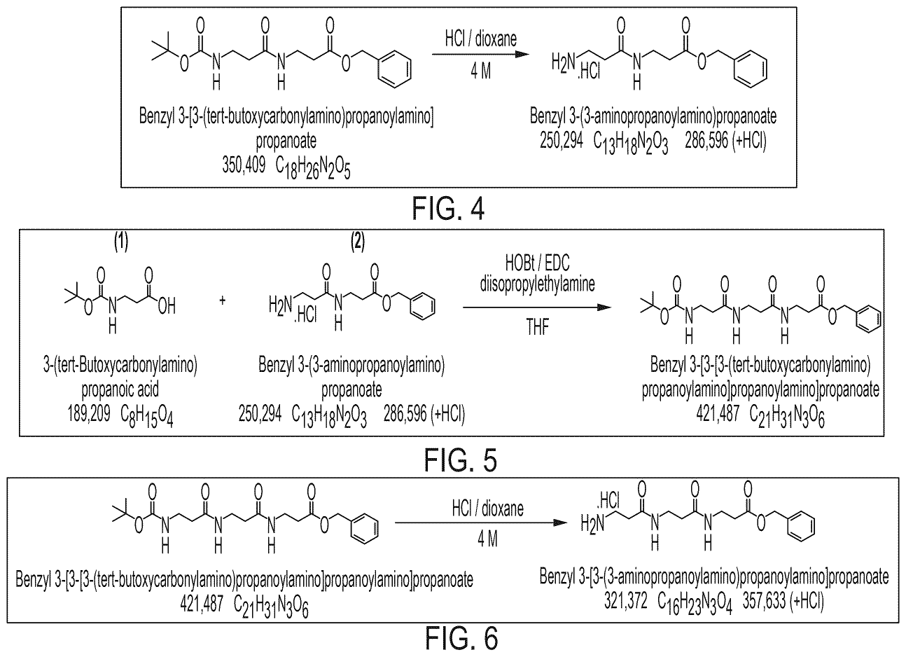

### Step 2: Synthesis of 3-aminopropionic acid benzyl ester hydrotosylate (H-βAla-OBn*pTosOH) (see FIG. 2)

A 1 L round bottom flask equipped with a Dean-Stark apparatus and a condenser is charged with 28.51 g 3-aminopropanoic acid (D-alanine), 199.64 mL benzyl alcohol, 65.25 g para-toluenesulfonic acid monohydrate-toluenesulfonic acid and 400 mL toluene. The reaction mixture is heated 5 hours under reflux and about 11 mL of water are separated, then about 200 mL toluene is distilled off. The solution is allowed to cool down, and at 45° C. to 50° C. 400 m L of diisopropyl ether is added slowly and the product starts to crystallize. The mixture is stirred for 1.5 hours and the temperature of the solution drops to 20° C.-25° C. and crystallization of the product goes to completion. The solid is filtrated, washed with diisopropyl ether, and dried in vacuo to yield 111.08 g product (0.316 mol; 98.8%), as para-toluenesulfonate salt.

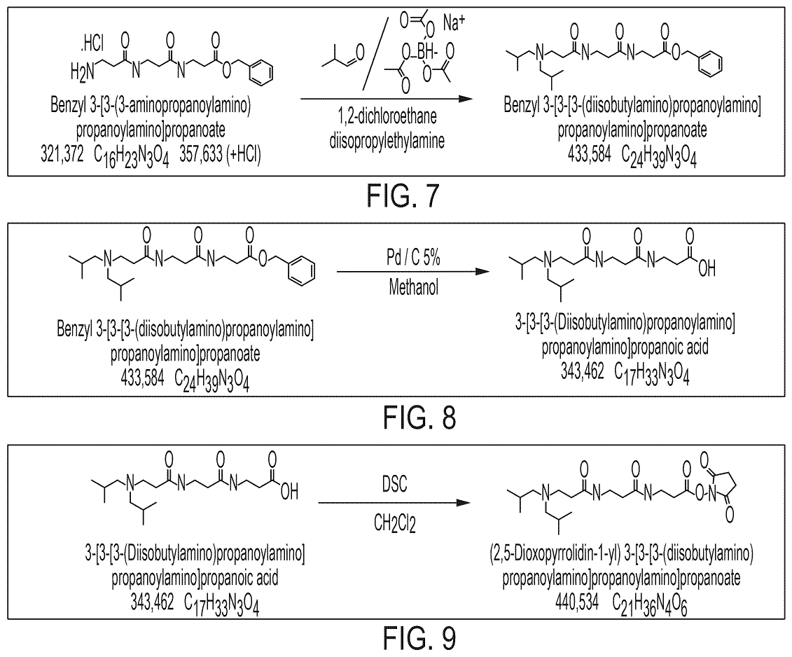

### Step 3: Synthesis of Benzyl 3-[3-(tert-butoxycarbonylamino)propanoylamino]propanoate (Boc-βAla-βAla-OBn) (see FIG. 3)

In a 1-L-two-neck flask, 18.21 g (1), 34.01 mL diisopropylethylamine (DIPEA), 35.14 g (2), 21.44 g 1-hydroxybenzotriazole monohydrate (HOBt) are added to about 200 mL tetrahydrofuran, whereby a suspension is formed. Add 24.92 g of 1-ethyl-3-(3-dimethyl amino-propyl)-carbodiimide hydrochloride (EDC) and the initial suspension goes into solution within 15-20 minutes and the temperature rises to about 30° C. Then stir for 7 hours at 25° C. and leave the mixture overnight at room temperature. Then, the reaction solution is concentrated on the rotary evaporator and the residue is dissolved in 400 mL ethyl acetate. It is washed two times with 500 mL saturated sodium hydrogen carbonate solution and once with 500 mL semi-saturated sodium chloride solution. The ethyl acetate phase is dried over MgSO4, filtered over silica gel, and concentrated. A solid white residue remains as product, to yield 32,54 g (92.86 mmol; 96,7%).

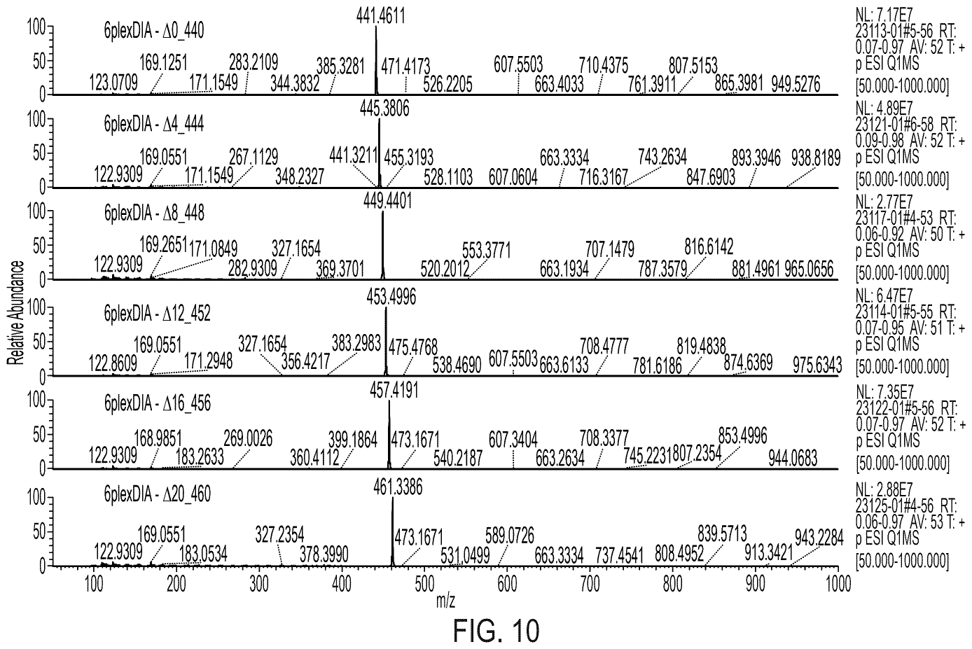

### Step 4: Synthesis of Benzyl 3-(3-aminopropanoylamino)propanoate hydrochloride (H-βAla-βAla-OBn*HCl) (see FIG. 4)

Dissolve 32.54 g Boc-βAla-βAla-OBn in 120 mL dichloromethane and add 60 mL 4M HCl in dioxane. Gas evolution starts and stirring is carried out for about 2 hours at 25° C. The reaction is monitored by thin-layer chromatography (dichloromethane:methanol 20:1).

After completion, the solution is concentrated on a rotary evaporator and dried, to yield the product quantitatively (26.61 g, 100%).

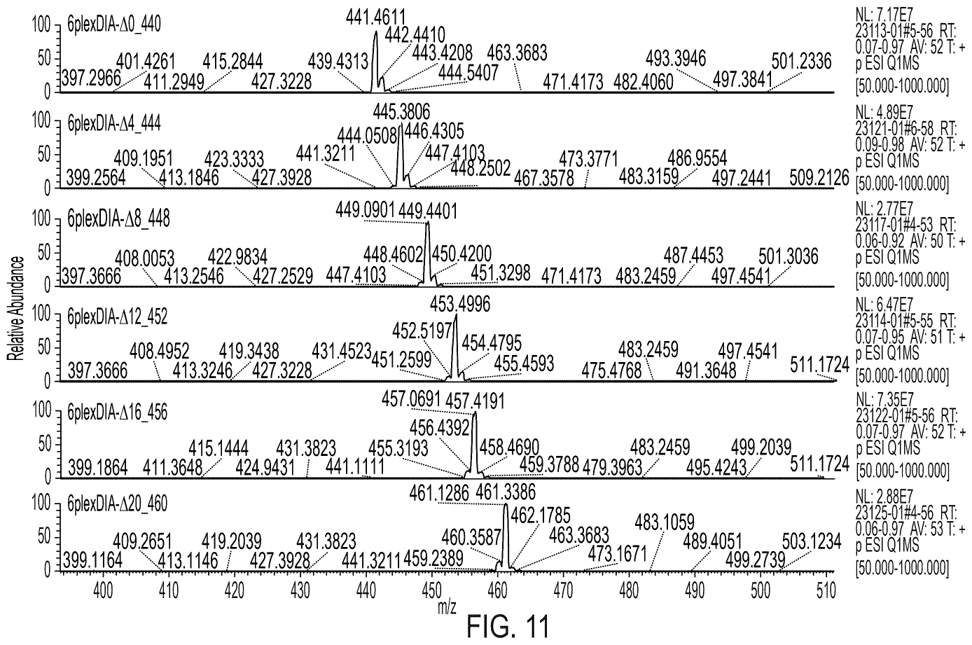

### Step 5: Synthesis of Benzyl 3-[3-[3-(tert-butoxycarbonylamino)propanoylamino]propanoyl amino]propanoate (Boc-βAla-βAla-βAla-OBn) (see FIG. 5)

In a 1-L-round-bottom flask, 17.57 g (1), 31.58 mL diisopropylethylamine (DIPEA), 26.61 g (2) and 19.91 g 1-hydroxybenzotriazole monohydrate (HOBt) are added to about 200 mL tetrahydrofuran, whereby a suspension is formed. Add 23.14 g of 1-ethyl-3-(3-dimethyl amino-propyl)-carbodiimide hydrochloride (EDC) and the initial suspension goes into solution within 20-30 minutes and the temperature rises to about 35° C. The mixture is then stirred for 5 hours at 25° C. and left overnight at room temperature. Then, the reaction solution is concentrated on the rotary evaporator and the residue is dissolved in 400 mL ethyl acetate. It is shaken out twice with 400 mL saturated sodium hydrogen carbonate solution and once with 500 mL semi-saturated sodium chloride solution. The ethyl acetate phase is dried over MgSO4 and concentrated. During concentration, the product crystallizes and yields 36.31 g (86.15 mmol, 92.8%).

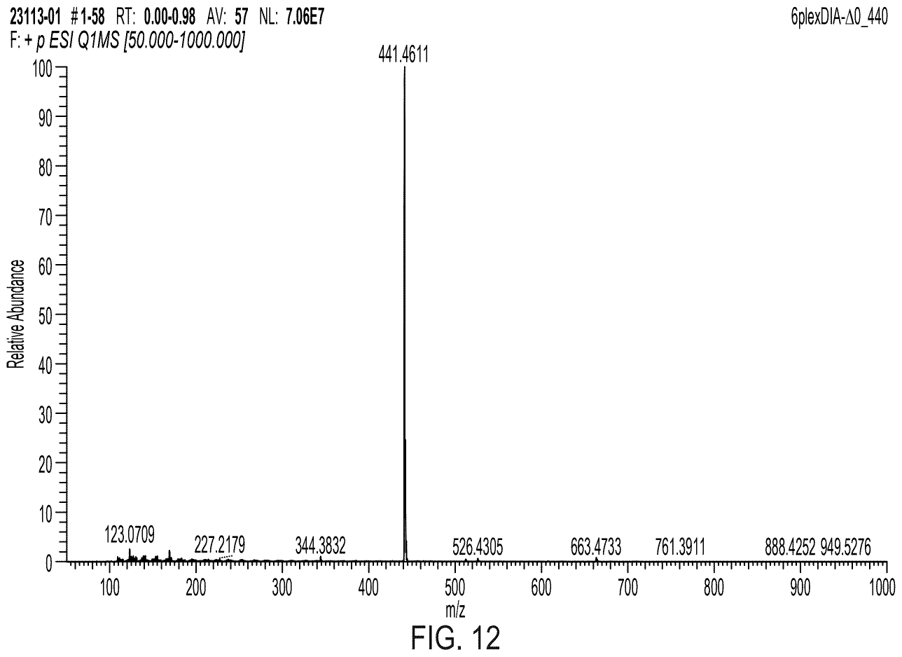

### Step 6: Synthesis of Benzyl 3-[3-(3-aminopropanoylamino)propanoylamino]propanoate hydrochloride (H-βAla-βAla-βAla-OBn*HCl) (see FIG. 6)

Dissolve 3.37 g Boc-βAla-βAla-βAla-OBn in 10.4 mL dichloromethane and add 5.2 mL 4M HCl in dioxane. Gas evolution starts and stirring is carried out for about 2 hours at 25° C. The reaction is monitored by thin-layer chromatography (dichloromethane:methanol 20:1). After completion, the solution is concentrated on a rotary evaporator and dried, to yield the product quantitatively (2.86 g, 100%).

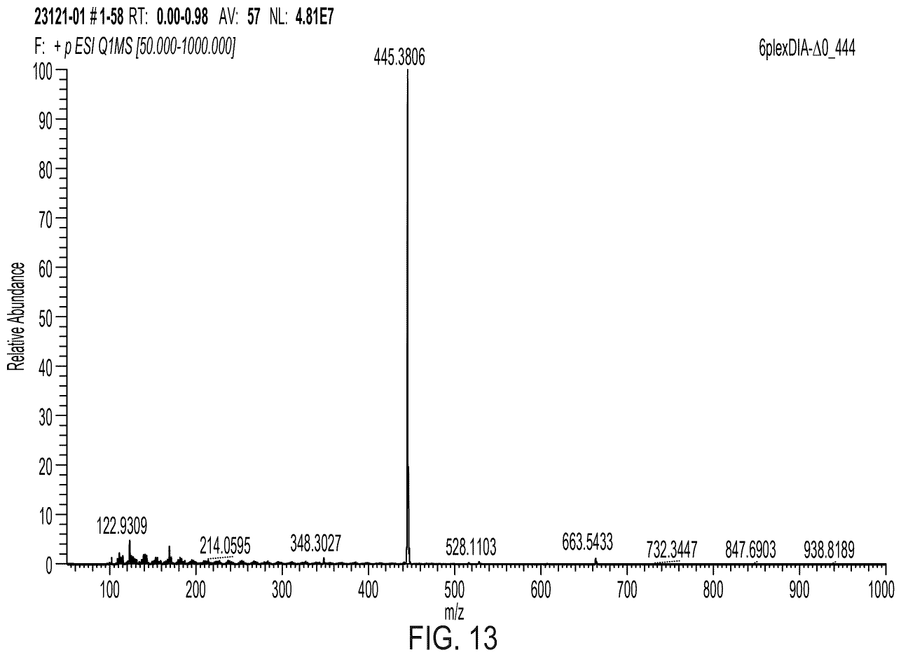

### Step 7: Synthesis of Benzyl 3-[3-[3-(diisobutylamino)propanoylamino]propanoylamino]propanoate (Diisobutyl-βAla-βAla-βAla-OBn) (see FIG. 7)

2.86 g H-βAla-βAla-βAla-OBn*HCl are stirred with 30 mL of 1,2-dichloroethane under argon. Then, 5.44 mL diisopropylethylamine and 1.83 mL isobutyraldehyde are added and stirred for 30 minutes to give a clear solution. Then, 6.78 g of sodium triacetoxyborohydride are added, the temperature rises to about 40° C. Stirring is continued overnight at 25° C. Then, remove about half of the 1,2-dichloroethane at the rotary evaporator and add 50 mL ethyl acetate to the residue. The organic layer is washed twice with 100 mL saturated sodium hydrogen carbonate solution and once with 100 mL semi-saturated sodium chloride solution, dried with MgSO4, filtered over silica gel with approx. 500 mL ethyl acetate and approx. 500 mL ethyl acetate:methanol 10:1 and concentrated. 3.35 g of the product is yielded as solid (7.72 mmol, 96.6%).

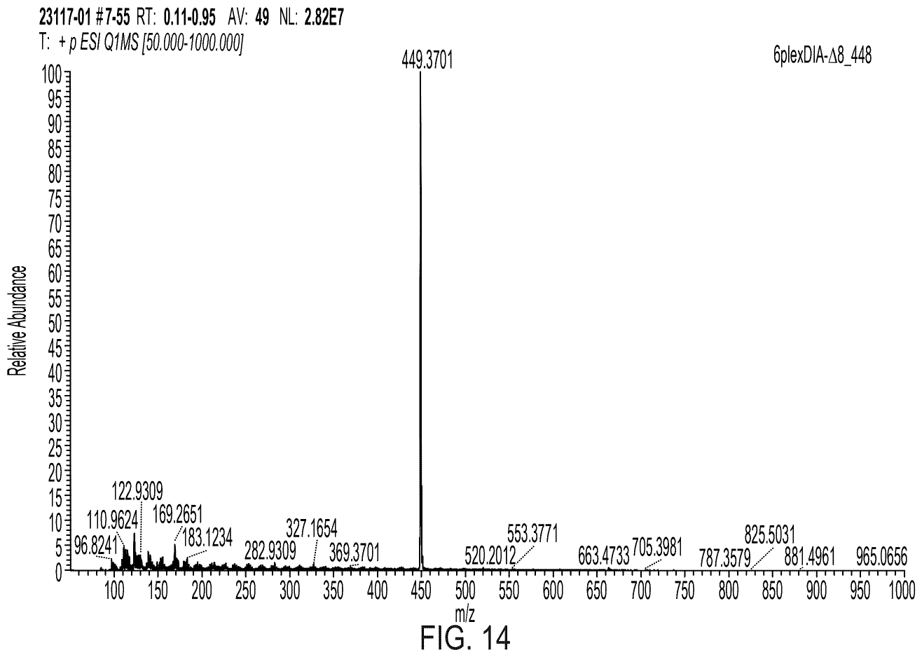

### Step 8: Synthesis of 3-{3-[3-(Diisobutylamino)propionylamino]propionylamino}propanoic acid (Diisobutyl-βAla-βAla-βAla-OH) (see FIG. 8)

0.17 g 5%-Pd/C catalyst and 20 mL methanol are placed under argon in a hydrogenation apparatus. Then, 3.35 g Diisobutyl-βAla-βAla-βAla-OBn, dissolved in 20 mL methanol are added, and hydrogenated at 25° C. until hydrogen uptake is complete. The reaction mixture is filtered through Dicalite filter aid to remove the Pd/C catalyst and the filter residue is washed with methanol. The filtrate is concentrated to dryness in vacuo. The oily residue is crystallized with ethyl acetate:diisopropyl ether 2:3 to yield 1.78 g of the product as white powder (5.19 mmol, 67%).

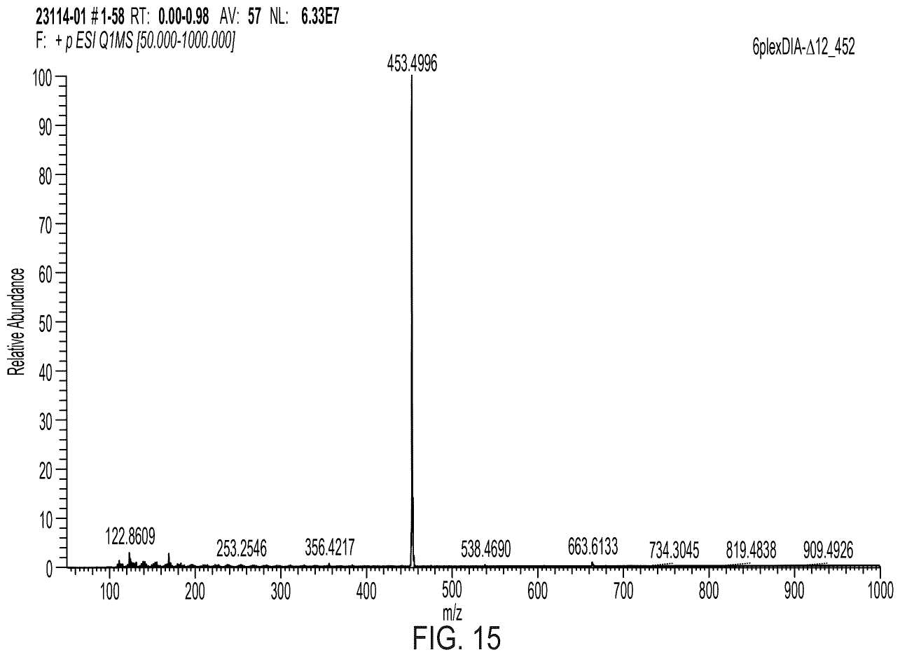

### Step 9: Synthesis of (2,5-Dioxopyrrolidin-1-yl)3-[3-[3-(diisobutylamino)propanoylamino]propanoylamino]propanoate (Diisobutyl-βAla-βAla-βAla-OSu) (see FIG. 9)

In a 100 mL flask, dissolve 1.29 Diisobutyl-βAla-βAla-βAla-OH in 25 mL dichloromethane at room temperature. Then, 1.25 g of N,N-disuccinimidylcarbonat (DSC) is added. The initial suspension goes into solution under CO2 evolution. After CO2 evolution is complete, the reaction mixture is stirred for 1 hour. Then, the reaction solution is diluted to 50 mL with dichloromethane and is shaken out three times with 40 mL each of saturated sodium hydrogen carbonate solution/saturated sodium chloride solution 4:1 and once with 40 mL semi-saturated sodium chloride solution. The dichloromethane phase is dried over MgSO4, filtered, and concentrated on the rotary evaporator.

The residue is dissolved in 5 mL ethyl acetate at about 65° C. During cooling down, the product crystallizes. The crystals are aspirated over a reverse frit under argon atmosphere, washed with ethyl acetate and diisopropyl ether and dried in vacuo at room temperature to yield 0.81 g of the product as white powder (1.84 mmol, 49%).

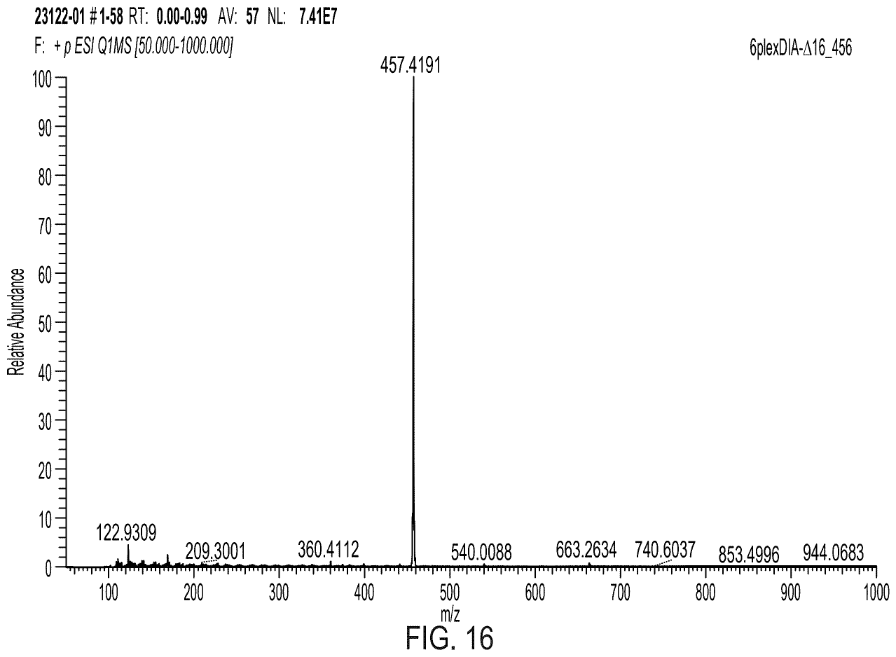

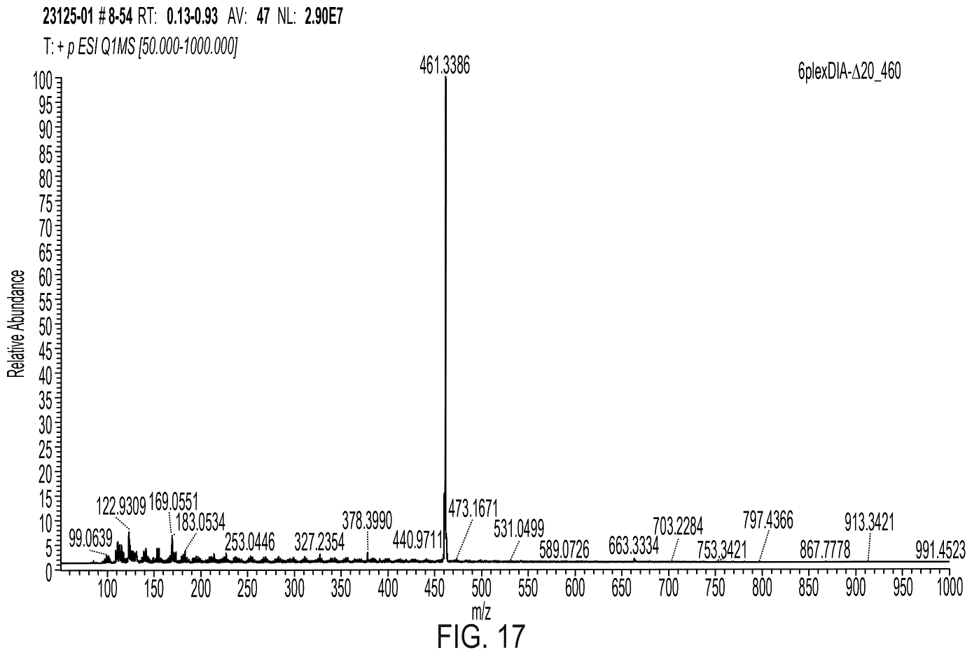

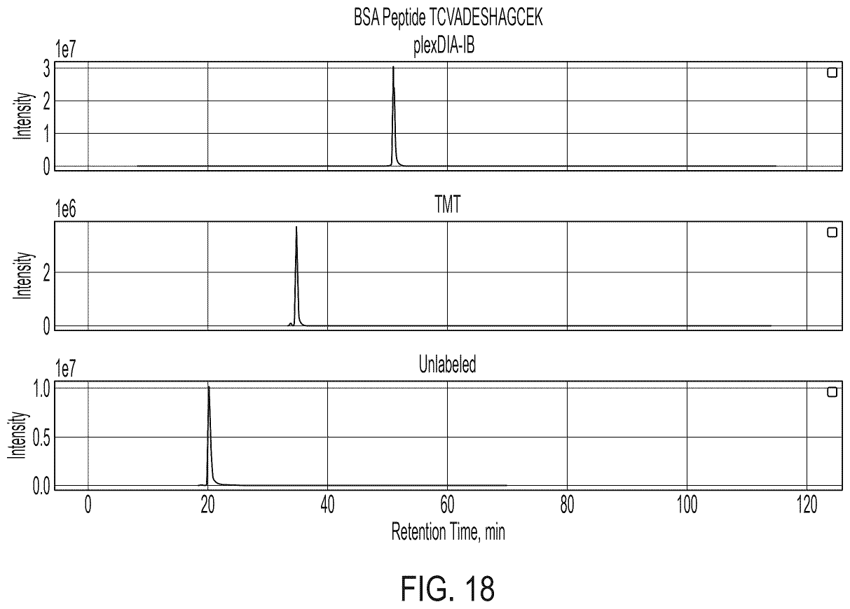

By using heavy-isotope labelled versions of β-alanine and isobutyraldehyde, it should be apparent to one of ordinary skill in the art that these can be substituted into the synthetic steps above to generate different versions with multiple different isotopic masses. FIGS. 10-17 illustrate the mass spectrum results for a set of 6 mass labels according to this example, with different isotopic substituents (i.e., as shown above by the chemical structures in Embodiment 1).

### Example 2: Synthesis of Additional 5 Isotopic Variants of (2,5-Dioxopyrrolidin-1-yl)3-[3-[3-(diisobutylamino)propanoylamino]propanoylamino]propanoate (Diisobutyl-βAla-βAla-βAla-OSu) (6plexDIA-Δ0_440)

After synthesis of undoped 6plexDIA-Δ0_440 we repeated synthesis with different precursors doped at different C and N positions as shown in embodiment 1. The synthesis conditions were identical in all other respects. The final products were infused into a TSQ Vantage mass spectrometer [Thermo Scientific] and the precursor mass and peak purity assessed (FIGS. 10-17). All tags showed the expected mass and exhibited very good purity.

### Example 3: Analysis of BSA Digest Using 6plexDIA-Δ0_440

To demonstrate protein labeling, we took 150 pmol of bovine serum albumin tryptic digest [New England Biolabs P8108S]. Peptides were labelled with 6plexDIA-Δ0_440 tag by 1 h incubation in 100 mM TEAB buffer. Labelling was quenched with addition of 0.25% hydroxylamine solution and labelled peptides were purified by StageTip and eluates dried to completion using a SpeedVac centrifuge. In parallel, we prepared unlabeled and Tandem Mass Tag® (TMT®)[Proteome Sciences plc] labelled samples for comparison.

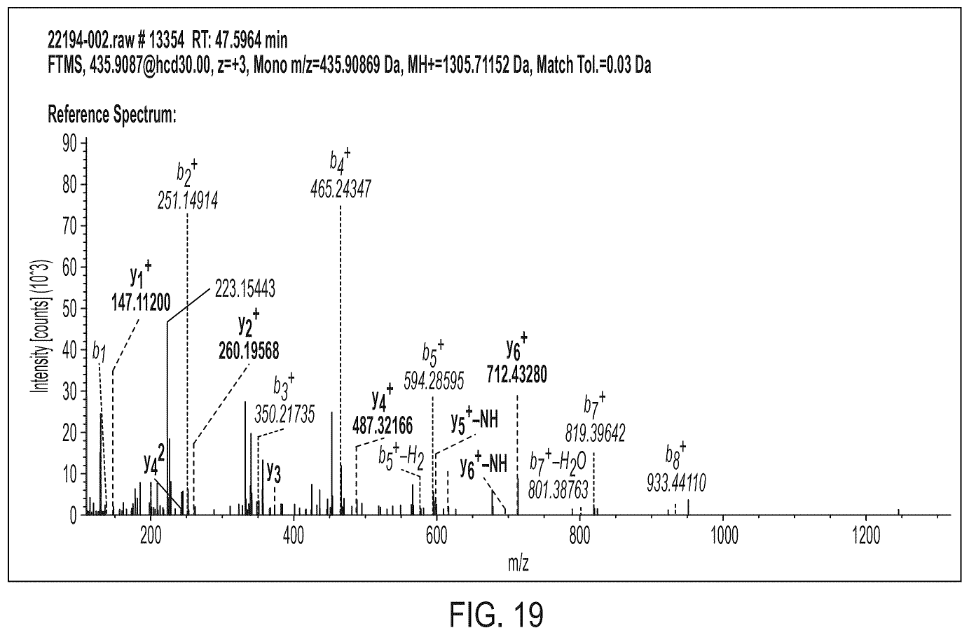

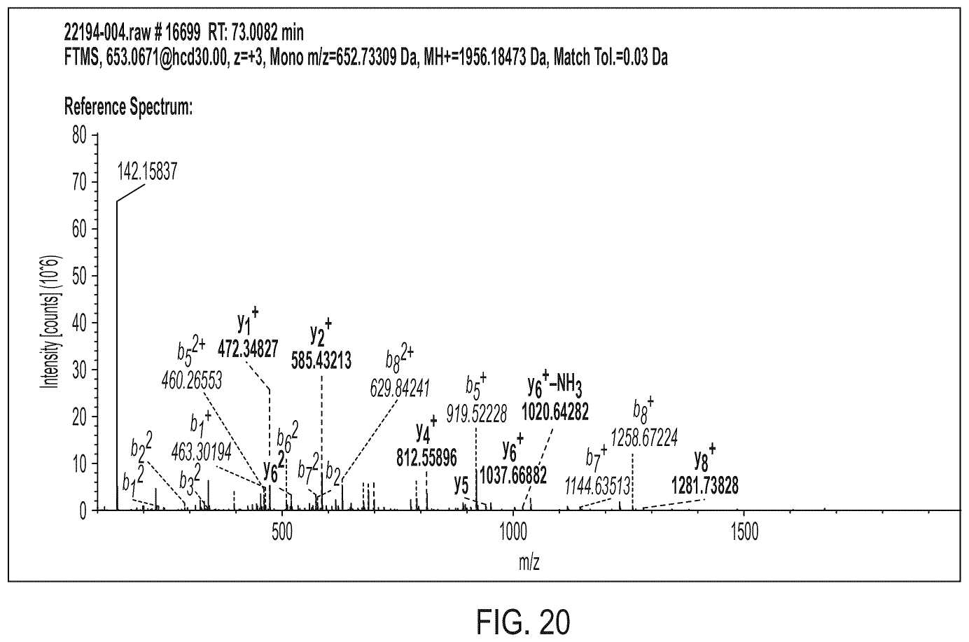

All three BSA digests were analyzed on an Orbitrap Fusion™ Tribrid™ mass spectrometer with on-line EASY-nLC™ 1200 UHPLC chromatography system [all Thermo Scientific] with spectra collected in data-dependent acquisition mode. As expected, the retention time of the 6plexDIA-Δ0_440 peptides was later than for TMT® or unlabeled equivalent peptide (FIG. 18). The MS/MS spectra of a typical BSA unlabeled peptide (FIG. 19) was compared with the 6plexDIA-Δ0_440-labeled equivalent (FIG. 20) and sufficient structural ions were observed to allow confident sequence assignment.

### Example 4: Analysis of HeLa Digests Using the 6Plex DIA Tags of Embodiment 1

Six replicate digests of the human HeLa cell line [Pierce Biotechnology] were independently labeled with one each of the 6plex DIA tags of Embodiment 1, essentially as described in Example 3. After quenching of the labeling reaction, the six digests were pooled to provide a single HeLa analytical sample. The HeLa analytical sample was analyzed by LC-MS/MS on an Orbitrap Exploris™ 480 with on-line EASY-nLC™ 1200 UHPLC system and FAIMS Pro source [all Thermo Scientific]. Peptides were eluted with an active acetonitrile gradient over 45 minutes. MS1 survey scans were collected at 120,000 resolution and MS/MS spectra acquired in DDA mode.

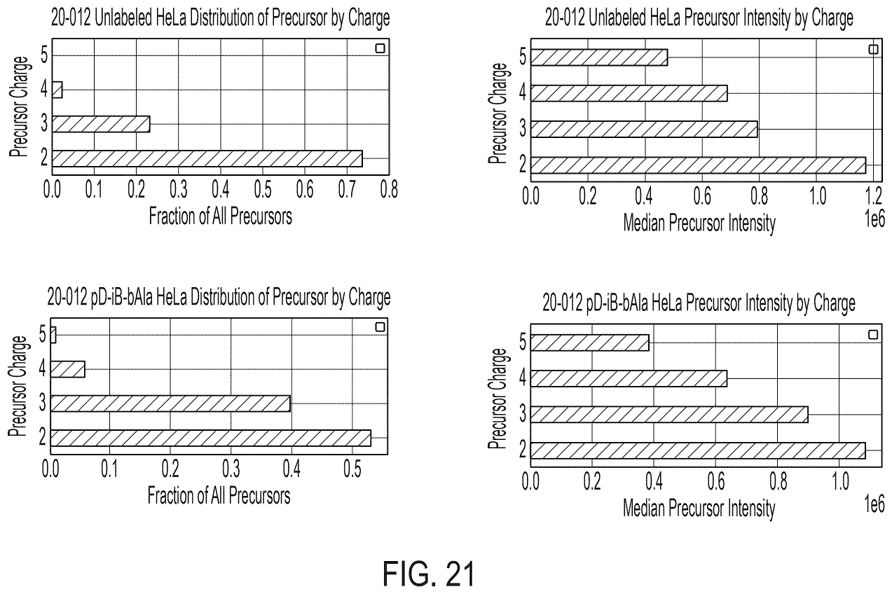

Analysis of data showed that compared with unlabeled digests, the 6plex DIA tags had a higher proportion of +3 ions and concomitant reduction in +2 ions, although with +2 ions still having the highest proportion. Similar increases were also seen for +4 and +5 ions, though their abundance remained relatively low. It was also notable that median precursor ion intensity for the +3 ions was higher in labeled digests compared with the unlabeled +3 ions. Examples for the unlabelled and labeled HeLa digests are shown in FIG. 21.

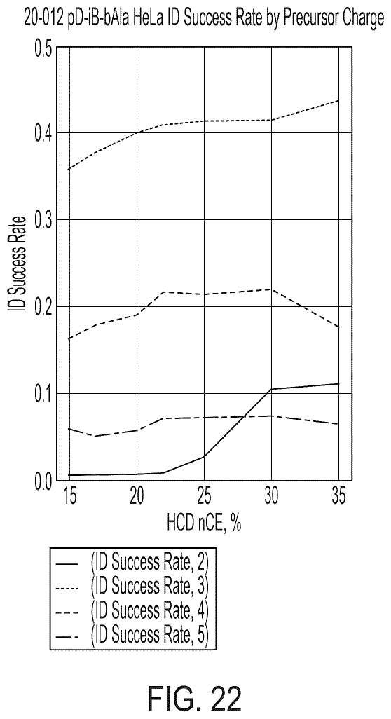

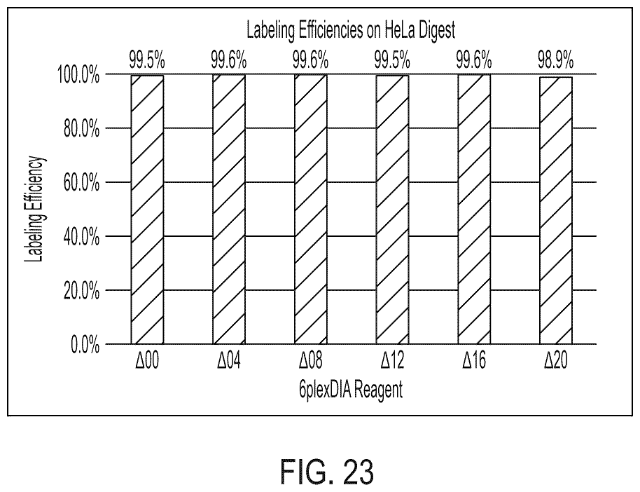

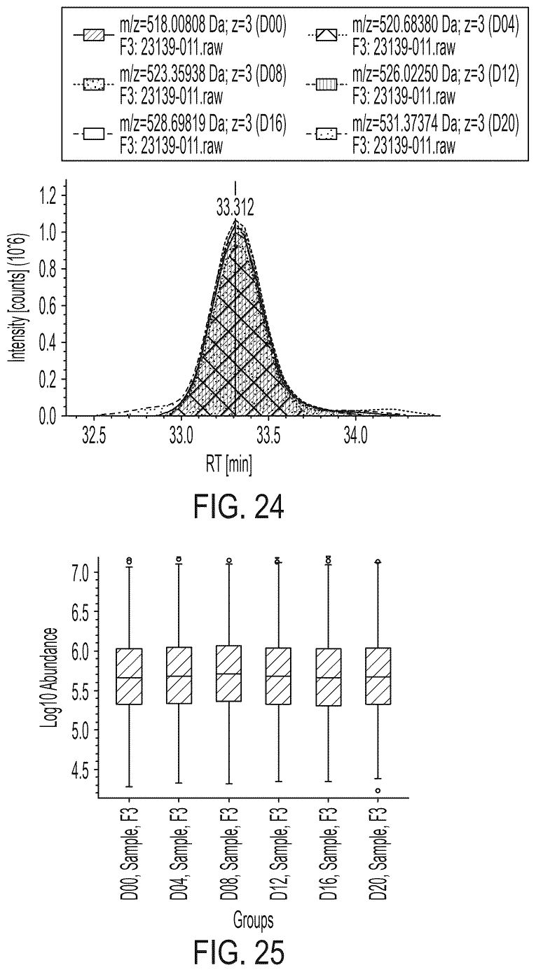

The +3 ions also had a higher identification success rate than all other charge states with a maximum of approximately 43% achieved with normalized collision energy of 35% (FIG. 22). All six tags showed very similar labeling efficiency in the HeLa digest with the percentage of labelled peptides ranging from 98.9 to 99.6% (FIG. 23). We also see that the six different tagged forms of peptides all co-elute perfectly (example for one peptide shown in FIG. 24) and the median precursor ion intensity for all six labeled digests is very similar (FIG. 25).

### Example 5: Analysis of BSA Digests Using the 6Plex DIA Tags of Embodiment 1

Six replicate digests of the BSA were independently labeled with one each of the 6plex DIA tags of Embodiment 1, essentially as described in Example 3. After quenching of the labeling reaction, the six digests were pooled to provide a single BSA analytical sample that was analysed by LC-MS/MS on an Orbitrap Exploris™ 480 mass spectrometer (Thermo Scientific) with on-line EASY-nLC™ 1200 UHPLC system. Peptides were eluted with an active acetonitrile gradient over 45 minutes. MS1 survey scans were collected at 60,000 resolution and MS/MS spectra acquired in DIA mode with 10 or 40 Da isolation windows.

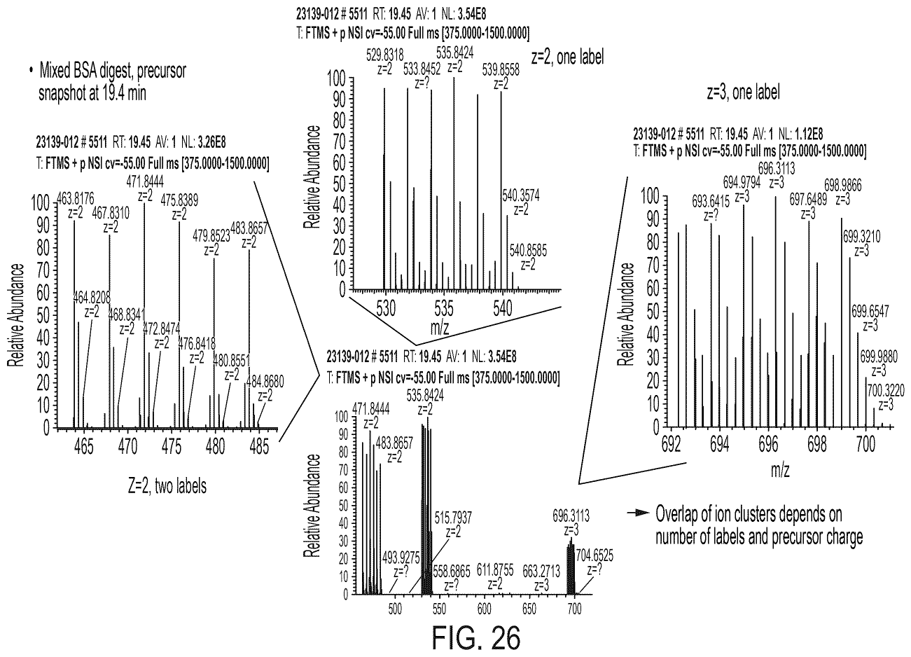

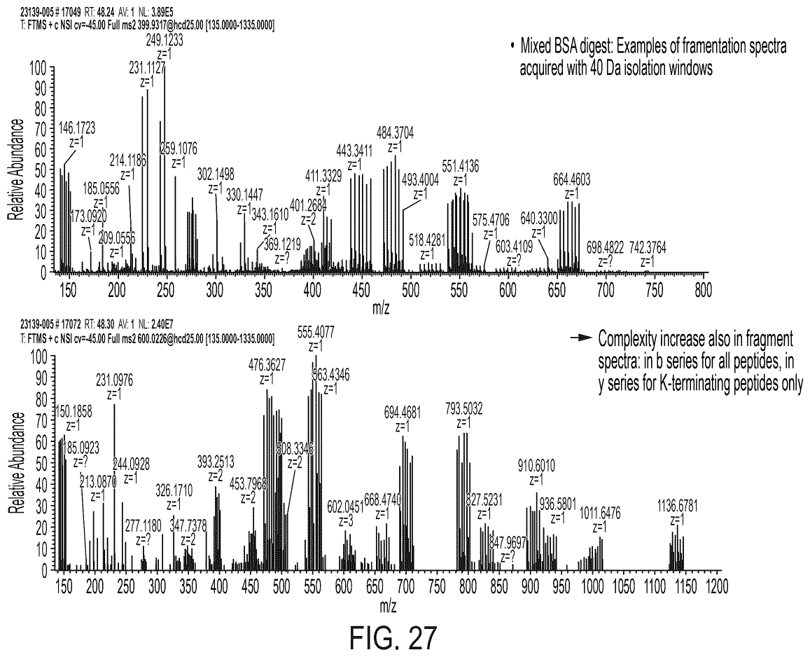

Analysis of data showed the expected increase in precursor ion intensity with isotopic envelope overlap being dependent on the charge state and number of DIA tags attached as shown for the survey scan at 19.4 minutes (FIG. 26). The expected complexity increase was also seen in the fragmentation spectra of a 40 Da isolation window MS/MS scan (FIG. 27). This was particularly apparent for peptides terminating in lysine where both b- and y-series ions showed higher complexity. For peptides terminating in any other amino acid, the y-ions did not increase in complexity whilst the matching b-ions showed the six variants due to presence of the DIA tags.

### Example 6—Analysis of HeLa Digest Spiked with Yeast Digest and Labeled with the 6Plex DIA Tags of Embodiment 1

195 μg of HeLa digests were spiked with six different amounts of yeast digest (0%, 2%, 3.5%, 5.8%, 9.7%, 15.6%) to generate six distinct samples. Samples were mixed individually with six 6plexDIA reagents (Table 1) at a final concentration of 16.5 mM and incubated for 1 hour at room temperature with shaking. The reactions were stopped by adding 0.25% hydroxylamine for 15 min at room temperature. 25% of each individually labelled peptide sample were purified by tandem solid phase extraction (Oasis HLB (Waters) and SCX (SP Sepharose Fast Flow, Sigma)) and eluates dried to completion using a SpeedVac centrifuge. 75% of the individually labelled peptides were pooled to generate one 6plex and 50% of this mixed 6plex was then purified by tandem SPE (HLB/SCX) as above and dried under vacuum.

TABLE 1
6plex DIA Tag Assignment to HeLa + Yeast digest samples

| | 1 | 2 | 3 | 4 | 5 | 6 |
|---|---|---|---|---|---|---|
| Sample | HeLa with_{—} | HeLa_with | HeLa with_{—} | HeLa_with | HeLa_with | HeLa with_{—} |
| | 0% yeast | 2% yeast | 3.5% yeast | 5.8% yeast | 9.7% yeast | 15.6% yeast |
| Tag | Δ00 | Δ04 | Δ08 | Δ12 | Δ16 | Δ20 |

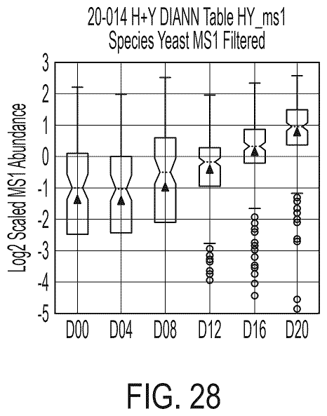

Peptides were analysed by LC-MS/MS on an Orbitrap Exploris™ 480 mass spectrometer (Thermo Scientific) with on-line EASY-nLC™ 1200 UHPLC system. Peptides were eluted with an active acetonitrile gradient over 110 minutes. MS1 survey scans were collected at 60K resolution and MS/MS spectra acquired in DIA mode with 10 Da isolation windows. The raw MS data file was searched in DIA-NN software using an in silico generated reference library. We identified 3,584 human and yeast proteins across the sixplex sample and saw the expected increase in precursor and fragment abundances of yeast peptides in line with the spiking concentration for samples with 5.8% yeast or greater (FIG. 28).

We claim:

1. A set of two or more isotopic mass labels, wherein each label comprises the formula: 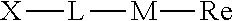
wherein:
X is a coding moiety having an exact mass,
L is a bond optionally cleavable by collision in a mass spectrometer,
M is a mass modifier,
Re is a reactive functionality for attaching the mass label to an analyte or the analyte, and
wherein X comprises the following general formula:

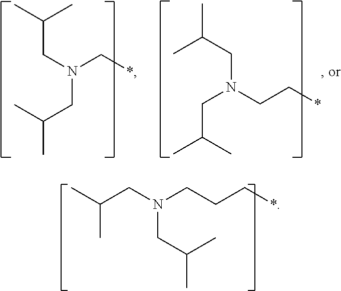

2. A set of two or more isotopic mass labels, wherein each mass label comprises the formula: 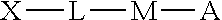
wherein:
X is a coding moiety having an exact mass,
L is a bond optionally cleavable by collision in a mass spectrometer,
M is a mass modifier, and
A is the analyte, and
wherein each mass label in the set has a different integer mass, and X comprises the following general formula:

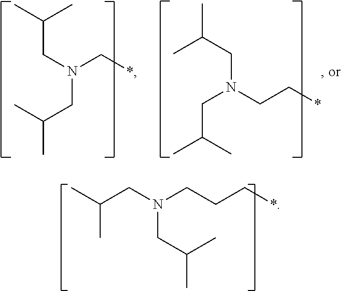

3. The set of two or more isotopic mass labels according to claims 1 or 2, wherein the mass modifier M has the following structure: 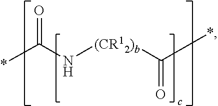
wherein:
each R₁ is independently H, a substituted or unsubstituted straight or branched C₁-C₆ alkyl group, a substituted or unsubstituted aliphatic cyclic group, a substituted or unsubstituted aromatic group or a substituted or unsubstituted heterocyclic group or an amino acid side chain,
a is an integer from 0-10,
b is at least 1, and
c is at least 1.

4. The set of two or more isotopic mass labels according to claims 1 or 2, wherein the mass modifier M has the following structure: 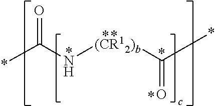
wherein:
each R₁ is independently H, a substituted or unsubstituted straight or branched C₁-C₆ alkyl group, a substituted or unsubstituted aliphatic cyclic group, a substituted or unsubstituted aromatic group or a substituted or unsubstituted heterocyclic group or an amino acid side chain,
a is an integer from 0-10,
b is at least 1,
c is at least 1, and
wherein * is an isotopic mass adjuster moiety and represents that oxygen is ¹⁸O, carbon is ¹³C, nitrogen is ¹⁵N or hydrogen is ²H, and wherein one or more * may be present.

5. The set of two or more isotopic mass labels according to any one of claims 1-4, wherein each mass label has the following structure: 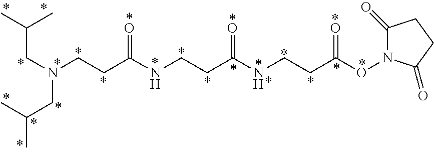 [SMILES: CC(C)CN(CCC(=O)NCCC(=O)NCCC(=O)ON1C(=O)CCC1=O)CC(C)C]
wherein * represents that oxygen is ¹⁸O, carbon is ¹³C, and nitrogen is ¹⁵N, and wherein one or more * may be present.

6. The set of two or more isotopic mass labels according to any one of claims 1-5, wherein the set comprises n=6 mass labels having the following structures:

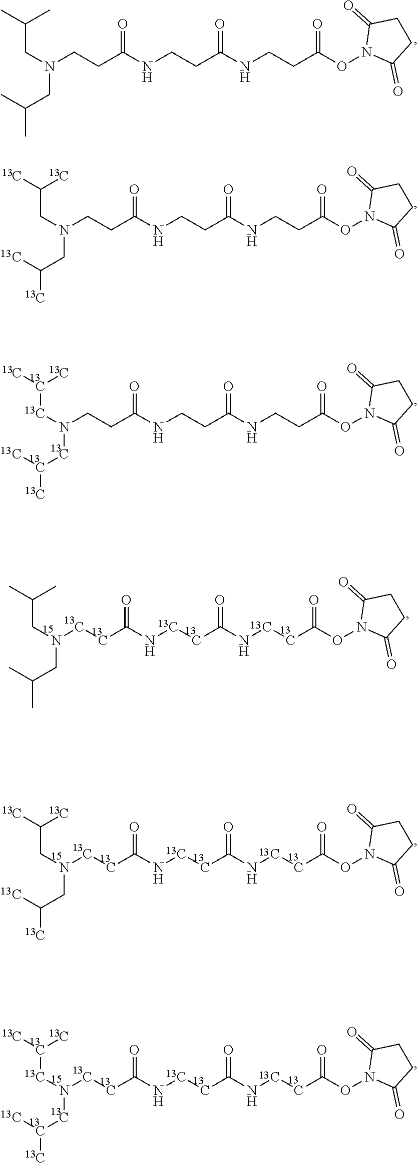 [SMILES: CC(C)CN(CCC(=O)NCCC(=O)NCCC(=O)ON1C(=O)CCC1=O)CC(C)C ; CC(C)CN(CCC(=O)NCCC(=O)NCCC(=O)ON1C(=O)CCC1=O)CC(C)C ; CC(C)CN(CCC(=O)NCCC(=O)NCCC(=O)ON1C(=O)CCC1=O)CC(C)C ; CC(C)CN(CCC(=O)NCCC(=O)NCCC(=O)ON1C(=O)CCC1=O)CC(C)C ; CC(C)CN(CCC(=O)NCCC(=O)NCCC(=O)ON1C(=O)CCC1=O)CC(C)C ; CC(C)CN(CCC(=O)NCCC(=O)NCCC(=O)ON1C(=O)CCC1=O)CC(C)C]

7. The set of two or more isotopic mass labels according to any one of claims 1-5, wherein the difference in exact mass between at least two of the mass labels is less than 100 millidaltons (“mDa”), less than 50 mDa, or less than 20 mDa.

8. The set of two or more isotopic mass labels according to any one of claims 1-5, wherein the difference in exact mass between at least two of the mass labels is 2.5 mDa, 2.9 mDa, 6.3 mDa, 8.3 mDa, 9.3 mDa, or 10.2 mDa.

9. A method of mass spectrometry analysis, comprising:
a) providing a plurality of samples, wherein each sample is differentially labelled with an isotopic mass label or a combination of isotopic mass labels, each selected from the set of two or more isotopic mass labels according to any one of claims 1-8;
b) mixing the plurality of labelled samples to form an analysis mixture comprising labelled analytes;
c) detecting the labelled analytes in a mass spectrometer;
d) isolating one or more labelled analytes in the mass spectrometer based on one or more isolation windows of ≥1 Daltons (“Da”) and then dissociating them to form labelled analyte fragments;
e) detecting the analyte fragments; and
f) identifying the one or more labelled analytes on the basis of the analyte fragments.

10. The method of claim 9, wherein the one or more isolation windows comprise an isolation window that is ≤10 Da.

11. The method of claim 9, wherein the one or more isolation windows comprise an isolation window that is ≤20 Da.

12. The method of claim 9, wherein the one or more isolation windows comprise an isolation window that is ≤50 Da.

13. The method of claim 9, wherein the one or more isolation windows comprise an isolation window that is ≤100 Da.

14. The method of any one of claims 9-13, wherein the dissociation in step d) is collision-induced dissociation in a mass spectrometer.

15. The method of any one of claims 9-14, wherein the dissociation is collision-induced dissociation in a mass spectrometer with a resolution of greater than 60,000 at a mass-to-charge ratio of 400, optionally a resolution of greater than 100,000 at a mass-to-charge ratio of 400, or a resolution greater than 250,000 at a mass-to-charge ratio of 400.

16. The method of any one of claims 9-15, wherein the labelled analytes may be identified on the basis of the MS2 spectrum of the labelled analyte fragments and quantified using the MS1 spectrum of the labelled analytes.

17. The method of any one of claims 9-16, wherein each isotopic mass label is resolved from all other tags in the set by at least 0.003 Da, by 0.006 Da, or at least 1 Da.
